(19) United States
(12) Patent Application Publication
(10) Pub. No.: US 2025/0089985 A1
(43) Pub. Date: Mar. 20, 2025

(54) Dynamic Assessment Of Airway Deformation

(71) Applicant: The Regents of the University of Colorado, a body corporate, Denver, CO (US)

(72) Inventors: Min-Hyung Choi, Denver, CO (US); Shane Transue, Denver, CO (US)

(21) Appl. No.: 18/293,258

(22) PCT Filed: Jul. 28, 2022
(86) PCT No.: PCT/US2022/038681

§ 371 Date: Jan. 29, 2024

Related U.S. Application Data
(60) Provisional application No. 63/226,678, filed on Jul. 28, 2021.

(51) Int. Cl. A61B 1/00; A61B 1/04; A61B 1/267; A61B 5/08; G06T 7/50

(52) U.S. Cl. CPC A61B 1/000094; A61B 1/000096; A61B 1/04; A61B 1/267; A61B 5/08; G06T 7/50; G06T 2207/10016; G06T 2207/10068; G06T 2207/10088; G06T 2207/20081; G06T 2207/30004

(57) ABSTRACT

In various implementations, technology is disclosed to dynamically assess airway deformation. A depth prediction model is used to estimate airway depth from monocular endoscopic images of an airway and to create a dynamic point cloud representation of the airway. The depth prediction model is trained using synthetic image and depth data derived from a 3D airway model. The dynamic point cloud representation of the airway can be used to visualize and quantify airway contours and to classify airway obstructions and behavior. The technology described provides objective measures of airway deformations and behavioral analysis of pulmonary related conditions.

### CROSS REFERENCE TO RELATED APPLICATIONS

This application is a national phase of International Application No. PCT/US2022/038681, filed Jul. 28, 2022; which claims the benefit of U.S. Provisional Patent Application No. 63/226,678, filed on Jul. 28, 2021, the contents of each of which are herein incorporated by reference in their entirety.

### BACKGROUND

Upper airway behavioral evaluation and modeling facilitate identifying, monitoring, and diagnosing common respiratory issues such as Obstructive Sleep Apnea (OSA) and airway collapse. OSA can be difficult to evaluate accurately and objectively due in part to the sporadic nature of apnea events and complex multi-level obstructions that can be position dependent (e.g., lateral vs supine vs prone, etc.), sleep stage dependent (e.g., NREM vs REM), or may depend on case severity. The implications of OSA include negative impacts on cardiovascular, neurocognitive, and metabolic system health that in severe cases may prevent consolidated sleep, leading to severe secondary health risks. Partial or complete OSA can also contribute to declines in pulmonary health including labored or paradoxical breathing, restless sleep due to apnea events, and mouth breathing.

Quantification of the degree of airway collapse or obstruction can be challenging when multi-level obstruction or partial collapse is present. Current diagnostic procedures for OSA often require an overnight sleep study, which documents Electroencephalogram (EEG) signals and breathing patterns (e.g., through polysomnography). Polysomnography provides a means to infer severity of passageway deformation but does not directly identify the static and dynamic source(s) of obstruction. Drug Induced Sleep Endoscopy (DISE), Computed Tomography (CT), and Cine Magnetic Resonance Imaging scans (Cine MRI scans) present valuable diagnostic information that can be incorporated within the DISE procedure to evaluate forms and sources of OSA, but each method has its own set of technical limitations, and practical challenges in widespread adoption for the evaluation of complex airway deformations.

For example, DISE may be used to visualize the upper airway for obstructions (e.g., while the patient is under sleep-mimicking sedation). However, DISE evaluation lacks a set of standards that defines how to consistently make measurements, thus making DISE observations subjective as they rely on manual inspection and categorical judging on a per patient basis, which can introduce unintended observational bias thus limiting the ability to objectively assess the degree of airway (e.g., airways, G.I. tract, etc.) collapse. As a result, medical experts viewing the same DISE video will frequently come to different conclusions based on their interpretation, which may hamper surgical planning and patient outcomes. CT and Cine MRI can provide functional scans of the airways and a quantitative evaluation of dynamic behaviors of airways but are often limited in use due to complexity (e.g., subject patients to radiation exposure, are not widely clinically available, etc.) and expense. Using stereography, lasers, and the like to obtain depth measurements of airways may be limited due to physical constraints of the airway, equipment expense, etc.

### SUMMARY

This summary is provided to introduce a selection of concepts in a simplified form that are further described below in the Detailed Description section. This summary is not intended to identify key features or essential features of the claimed subject matter, nor is it intended to be used as an aid in determining the scope of the claimed subject matter. Additional aspects, features, and/or advantages of examples will be set forth in part in the description which follows and, in part, will be apparent from the description or may be learned by practice of the disclosure.

Non-limiting examples of the present disclosure describe systems, methods, and devices that improve the accuracy and quality of assessing airway deformation. In an implementation, a depth classification system is trained to classify airway deformation by identifying one or more characteristics of a video image and linking the one or more characteristics of the video image with fluid flow data. The one or more characteristics of the video image may include light variations between pixels, scalar and/or vector descriptions produced for the video image, and the like. The link may be based on a position along the length of the airway, one or more behaviors observed in the video image, etc. The fluid flow data may comprise a rate of flow, and the link may indicate a rate of flow at the position along the length of the airway.

In another implementation, a deep learning model is employed that incorporates trained models with DISE imaging procedures to perform objective. Behavioral scans may be integrated with monocular images of an airway to generate 3D surface models calibrated to obtain measurements in physical units.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Non-limiting and non-exhaustive examples are described with reference to the following figures.

FIG. 1 illustrates an operational architecture in an implementation of enhanced classification technology for assessing airway through the trained machine learning framework.

FIG. 2 illustrates processes for training a machine learning model for assessing airways and for assessing airways according to the model in an implementation.

FIG. 3A illustrates an exemplary cross-sectional airway measurement between two different breathing states from a Cine-MRI scan in an implementation.

FIG. 3B illustrates a process of virtualized airway modeling for augmenting network model training data through synthesized imaging in an implementation.

FIG. 3C illustrates a functional set of training data used to build the correlative model between RGB image sequences from DISE video data with corresponding depth images in an implementation.

FIG. 4A illustrates an operational overview of a correlative data processing pipeline for the estimation of dynamic 4D airway surface models in an implementation.

FIG. 4B illustrates an airway evaluation pipeline leading from DISE video data paired with Cine-MRI scan data to produce 4D airway models in an implementation.

FIG. 5A illustrates synthetic video sequence data and comparative ground-truth data of the depth data used to define structural and behavioral analysis through 3D modeling in an implementation.

FIG. 5B illustrates depth estimations on low quality DISE video data sequences in an implementation.

FIG. 5C illustrates depth estimations on synthetic video sequence data in an implementation.

FIG. 6 illustrates an airway classification process and a classification model training process in an implementation.

FIG. 7A illustrates representations of a DISE Frame and a synthetic frame paired with a synthetic depth frame in an implementation.

FIG. 7B indicates a representation of a video sequence formed from paired frames in an implementation.

FIG. 7C illustrates a representation of a data set including paired video sequences according to a parameter set in an implementation.

FIG. 8 illustrates a depth prediction model architecture in an implementation.

FIG. 9 illustrates airway contours extracted from an MRI Scan in an implementation.

FIG. 10A illustrates airway contours superimposed on RGB images in an implementation.

FIG. 10B shows a comparison of airway contours with airway wall displacement mapping in an implementation.

FIG. 11 shows an airway classification process in an implementation.

FIG. 12 illustrates a computing system suitable for an implementation of enhanced classification technology disclosed herein, including any of the applications, architectures, elements, processes, and operational scenarios and sequences illustrated in the Figures and discussed below in the Technical Disclosure.

FIG. 13 illustrates an operational architecture in an implementation of enhanced classification technology for assessing airway deformation and associated direct measurements obtained through the trained machine learning framework. This incorporates images obtained from DISE monocular video sequences, comparison between ground-truth and predicted depth samples generated, and the resulting change in cross-sectional area of the airway.

FIG. 14A illustrates a classification process in an implementation that describes the translative process of generating synthesized DISE videos from 3D printed airway models. Tissue samples and 3D printed modeling are used to supplement real-world synthesized in vitro training data for model generation. The 3D model is generated from scan sourced data slices, provided to a 3D printer, and then evaluated in training data for depth estimations that are directly compared with the real-world printed model.

FIG. 14B illustrates a classification process in an implementation that correlates depth descriptors of DISE recording sequences with structural descriptions of the airway for fine-grained measurement and classification operations. The measurements and classification data are then used to evaluate structural deformation changes within the airway for condition diagnosis and surgical planning.

FIG. 15A illustrates the cross-sectional airway measurement between two different breathing states from a Cine-MRI scan. This axial view provides a quantitative description of the airway opening that can be used to evaluate airflow through behavioral analysis. This quantitative data is linked with DISE acquired video data and trained network model to obtain measurements directly from monocular videos of the airway.

FIG. 15B illustrates a process of virtualized airway modeling for augmenting network model training data through synthesized imaging. Virtualized scan data is used to define structural airway models that are used for generating images that are correlated with ground-truth depth values provided through ray-casting algorithms.

FIG. 15C illustrates a functional set of training data used to build the correlative model between RGB image sequences from DISE video data with corresponding depth images. Large datasets are formulated for different levels of estimation accuracy based on training data sources that range from synthetic data, 3D printed models, in vitro modeling, and DISE video.

FIG. 16A illustrates the operational overview of the correlative data processing pipeline for the estimation of dynamic 4D airway surface models. Described within this process is the collection of paired Cine-MRI scan data, DISE video sequences, and depth images assembled into datasets used to train the model for depth predictions and airway surface reconstruction. From the resulting airway model, dynamic structural changes and deformations can be quantitative evaluated through model space coordinates defined by the paired Cine-MRI data.

FIG. 16B illustrates an airway evaluation pipeline leading from DISE video data paired with Cine-MRI scan data to produce 4D airway models. This introduces a diagnostic system that can be used to provide conditional evaluation through video-based airway measurements and behavioral analysis.

FIG. 17A illustrates DISE video sequence data and comparative ground-truth data of the depth data used to define structural and behavioral analysis through 3D modeling. Differences between ground-truth (training, synthesized, model, etc.) data and predictions are shown for the provided input image data.

FIG. 17B illustrates depth estimations on low quality DISE video data sequences. In vivo image data is used to predict the structural characteristics of the airways for evaluation.

### DETAILED DESCRIPTION

Various embodiments will be described in detail with reference to the drawings, wherein like reference numerals represent like parts and assemblies throughout the several views. Reference to various embodiments does not limit the scope of the claims attached hereto. Additionally, any examples set forth in this specification are not intended to be limiting and merely set forth some of the many possible embodiments for the appended claims.

Technology is disclosed herein for dynamically assessing airway deformation. In an embodiment, a depth classification system generates one or more behavioral models that enable quantitative contour measurements of airways. The one or more behavioral models may be created by integrating image-to-surface modeling, deep learning approaches of Machine Learning (ML), and DISE procedures. The one or more behavior models may then be employed by the depth classification system to estimate the contours and/or mean volumes of airways in video images (e.g., live DISE, monocular endoscopic video sequence, etc.).

Airways include passages for air, food, and liquid. Examples of airways include, but are not limited to, nasal cavity, nasopharynx, oropharynx, hypopharynx, esophagus, larynx, trachea, gastrointestinal (GI) tract, and the like.

The VOTE (velum, oropharynx, tongue base, and epiglottis) scoring system is used as a surgical framework in endoscopic OSA evaluation. The VOTE scoring system is a classification system for analyzing four levels of severity at five anatomical obstruction sites. The OSA evaluation may include assessment of the upper airway (e.g., from the choanae to the larynx, may include the trachea, etc.) to identify complex cases of OSA. The OSA evaluation may incorporate the velum, tongue base, hypopharynx, and larynx. The VOTE scoring system is subjective; thus, making an accurate evaluation of determining subtle reductions in airflow, including relative measurements, challenging to infer from endoscopic video.

The systems, methods, and devices described herein provide an improved means for measuring airway deformation that addresses existing diagnostic gaps by at least providing a truly objective, standardized, and quantitative evaluation of deformation in an airway (e.g., during DISE). One technical effect includes improving airway contour cross-sectional measurements to a sub-centimeter level where the measurement error (ϵ) of the airway diameter (d) can be defined as ϵ(d)<5.0 mm. Another technical effect includes a reduction in the requirement for advanced scans (e.g., CT, Cine MRI, etc.), which in turn reduces cost and increases the benefit to a broader patient base by at least improving objective prognosis tracing. Advanced scans may provide quantitative measurements of fluid flow restrictions and airway dynamics, but tend to be operationally expensive, subject patients to radiation exposure, and are not widely clinically available. By employing synthesized data for depth-image training, the depth classification system provides 4D reconstruction of airways (e.g., internal structures), images of which may be obtained through endoscopic devices.

Existing methods of airway evaluation are limited in terms of overcoming occlusions and dynamic reconstructions and have limited direct integration with DISE procedures. In contrast with the existing approaches, the depth classification system disclosed herein provides a means for obtaining consistent quantitative measurements and may leverage Generative Adversarial Network (GAN) depth predictions with Cine MRI behavioral data to provide better models for airway evaluations, which in turn provides more accurate diagnostics when compared with the subjective nature of current airway assessments.

The technology disclosed herein may be employed for endoscopic procedures including nasopharyngolaryngoscopy (NPL), used in an initial evaluation with no sedation, bronchoscopy for the lower airways and lungs, and the like.

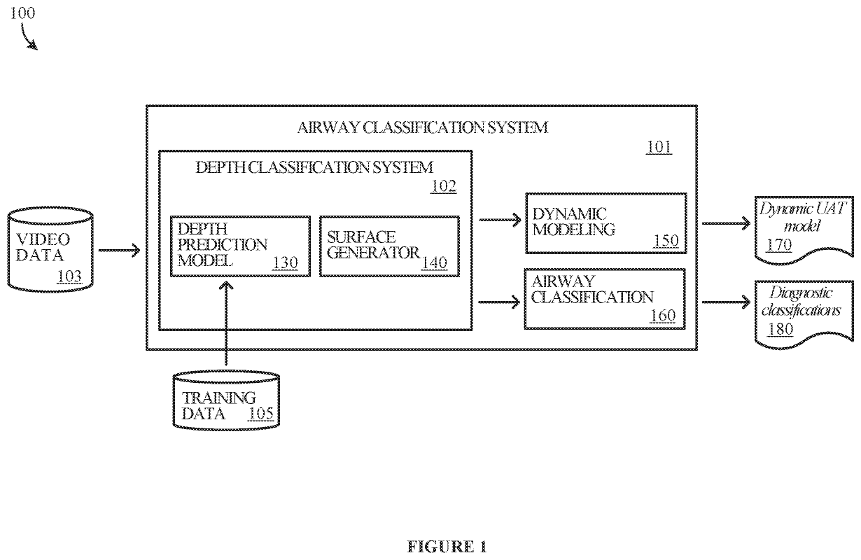

FIG. 1 illustrates operational architecture 100 in an implementation of enhanced classification technology for assessing airway deformation. Operational architecture 100 includes airway classification system 101 comprising depth classification system 102, video imagery data 103, and training data 105. Depth classification system 102 employs depth prediction model 130 and surface generator 140. Video imagery data 103 may include DISE video, monocular video sequences, and the like. Training data 105 may include synthetic models, 3D printed models, fluid flow evaluations using computational fluid dynamics, general fluid flow data, etc. Training data 105 may also include endoscopic video procedures linked to Cine MRI, 3D reconstructions of the airway, airway surface model datasets (e.g., GAN model datasets), etc.

Depth classification system 102 includes depth prediction model 130 which generates depth information of the airway corresponding to video imagery data 103. In an implementation, depth prediction model 130 receives video imagery data 103 in the form of RGB (Red, Green, Blue) images and outputs a predicted depth image. The RGB images may be, in an implementation, endoscopic RGB frames extracted from video imagery data 103. The predicted depth image may comprise an array of pixels corresponding to an RGB image, each pixel corresponding to a pixel of the RGB image and comprising a value representative of depth. In an implementation, the array of pixels of the predicted depth image comprises a grey-scale image of the predicted depth for a corresponding RGB image.

To train depth prediction model 130 for use in predicting depth information for the RGB imagery data, a three-dimensional structural model is generated based on MRI data which provides structural information and endoscopic imagery data which provides surface information for the 3D structural model. From the 3D structural model of the airway, a training dataset is generated comprising synthetic image data and corresponding synthetic depth frames comprising depth data.

In an implementation, depth prediction model 130 is based on deep learning or machine learning, for example, a self-supervising learning implementation such as GAN, trained to generate predicted depth information from video imagery data 103. The depth prediction model used by depth prediction module 130 is trained on datasets comprising synthetic DISE imagery data with synthetic depth data derived from a 3D structural model generated from Cine-MRI data and endoscopic or DISE (drug-induced sleep endoscopy) imagery data.

The predicted depth image generated by depth prediction model 130 includes a single depth value “D” for each pixel of the corresponding RGB image, where D represents a distance between the monocular camera and the airway wall surface. The predicted depth image may be an endoscopic predicted depth image (when the input to the model is an endoscopic RGB image) or a synthetic predicted depth image (when the input to the model is a synthetic RGB image).

Surface generator 140 receives the depth prediction data from depth prediction model 130 along with video data 103 to generate 3D surface mesh construction of a UAT or of a portion of the UAT. The 3D surface mesh representation comprises, in an implementation, a point-cloud surface model. In an implementation, surface generator 140 forms a 3D point which aggregates color (RGB), a surface normal vector, and a position in three-dimensional coordinates. The 3D point-cloud may represent a region or patch of the surface of the UAT.

Depth classification system 102 outputs a sequence of surface constructions to dynamic modeling module 150 which generates a dynamic or 4D model of the UAT which may be viewed to demonstrate UAT dynamic behavior. Depth classification system 101 also outputs the surface mesh construction to airway classification module 160 which quantifies various physical parameters of the UAT or of UAT contours at specified locations, but also classifications of obstructions or of airway behavior.

### Depth Prediction Model Training

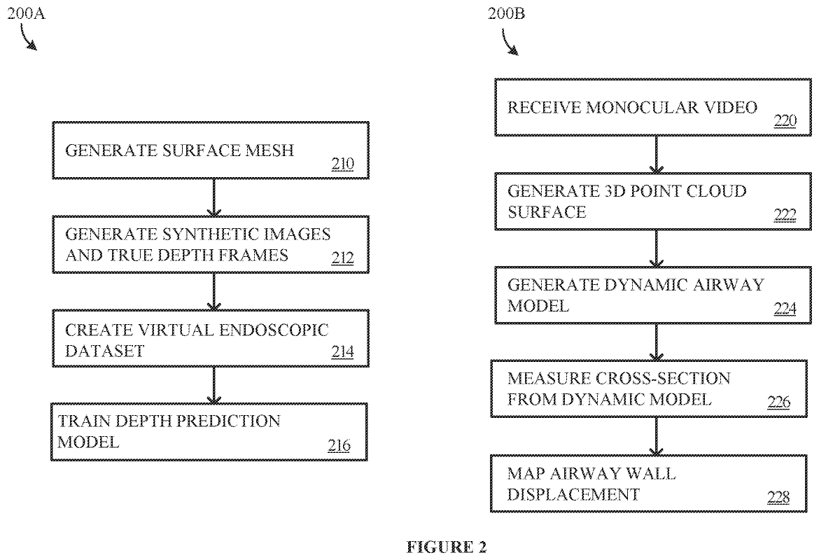

In some implementations, the depth prediction module of the depth classification system is trained using the depth prediction model training process, an implementation of which is shown in process 200A of FIG. 2. Process 200A results in a trained depth prediction model that is able to predict depth in airways from monocular endoscopic images.

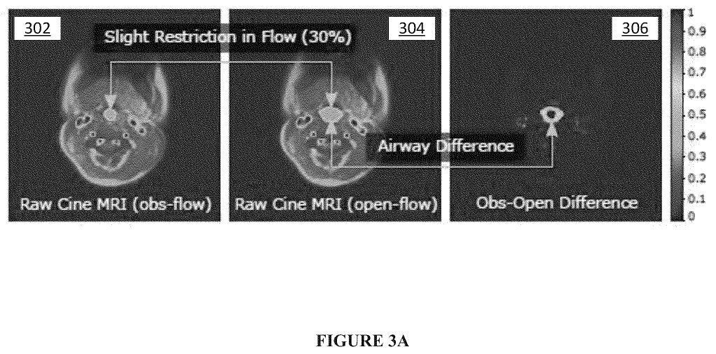

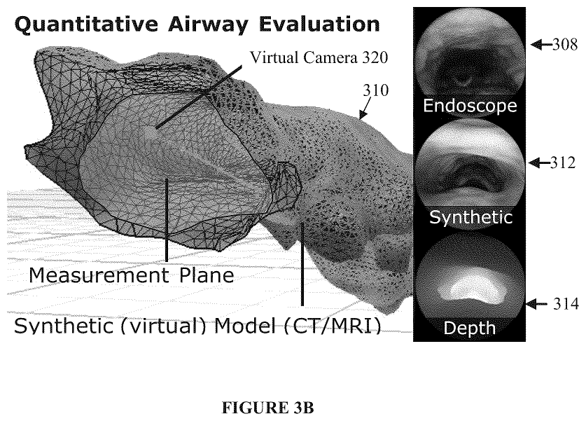

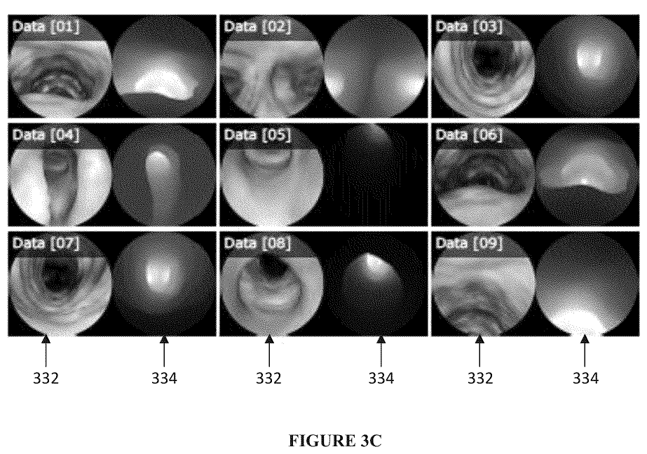

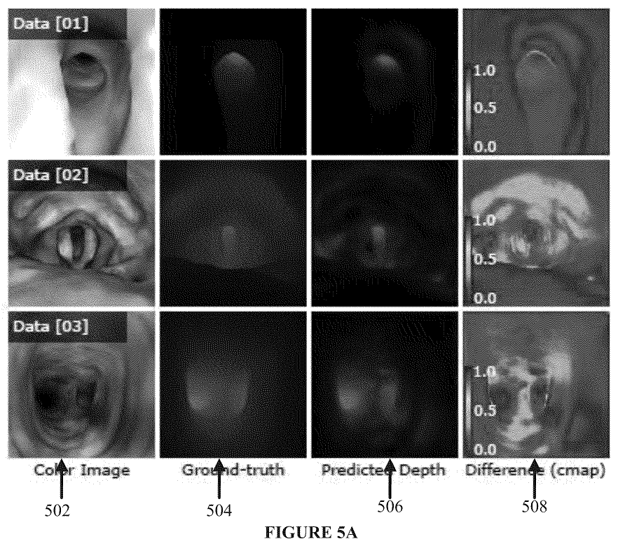

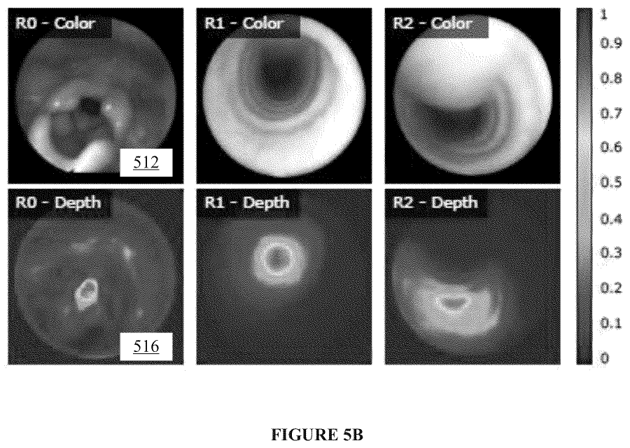

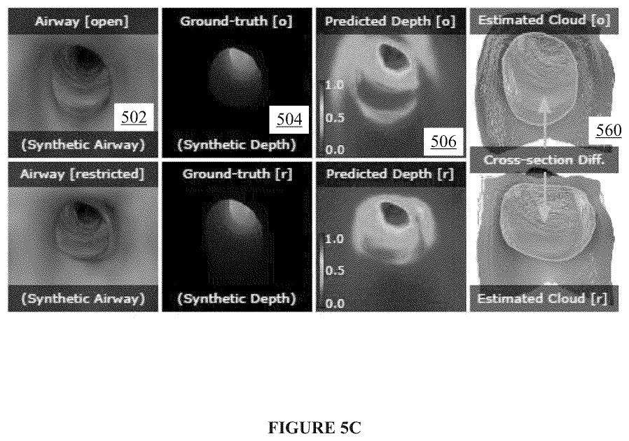

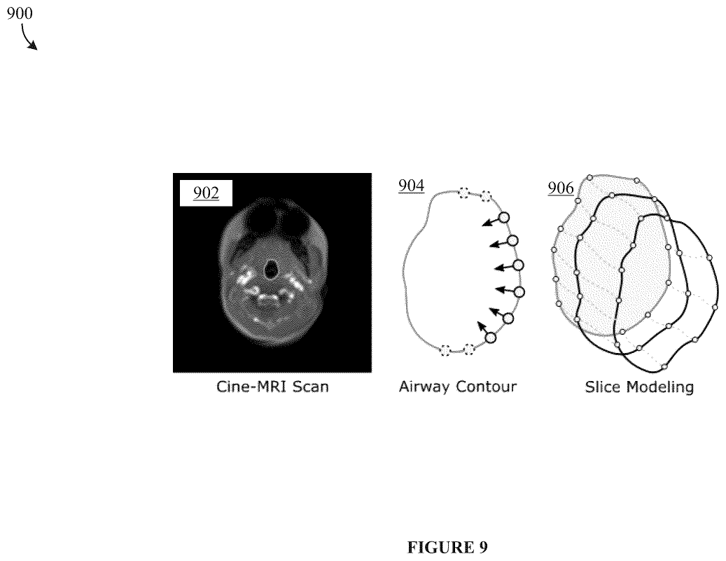

In step 210 of the depth prediction model training process 200A, a surface mesh is generated, an example of which is surface mesh 310 in FIG. 3B. The surface mesh is a three-dimensional virtual or synthetic representation of an airway, for example the Upper Airway Tract (UAT). The surface mesh is used to generate synthetic images, such as synthetic images 502 of FIG. 5A for training of the depth prediction model. Generating the surface mesh comprises extracting contours from Cine-MRI data and stacking contours axially and extracting surface information (e.g. color and texture information) from real endoscopic imagery. The surface mesh may be a dynamic surface mesh to represent deformations in an airway over time. This process is discussed in more detail in relation to FIG. 9 below.

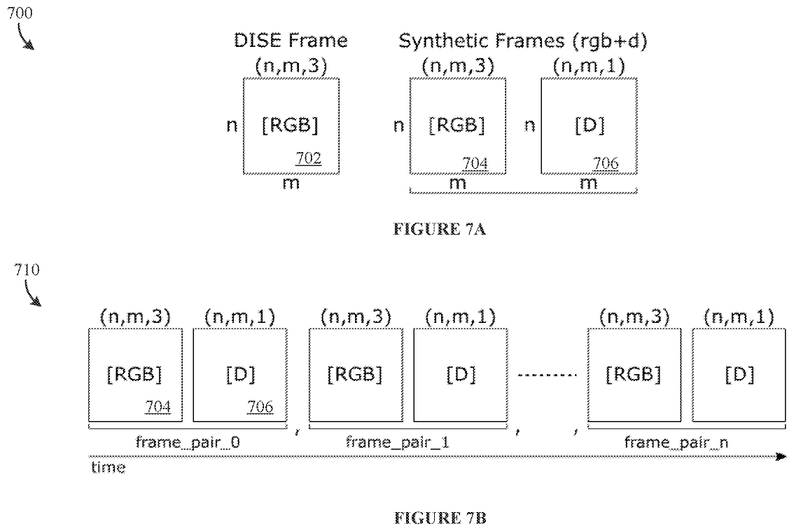

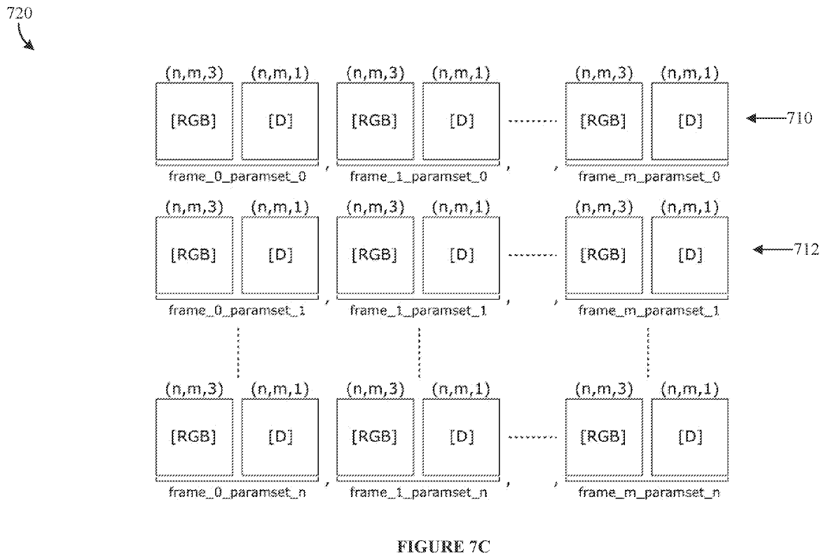

In step 212, synthetic images of the airway, such as synthetic images 502 in FIG. 5A, and ground-truth depth images, such as ground-truth depth images 504 of FIG. 5A, are created from the surface mesh. The synthetic images simulate what an image of the airway would look like if the image was taken from a camera (e.g., virtual camera 320 of FIG. 3B) at a specific position within the surface mesh model of the airway. Each pixel of the ground-truth depth images represents a distance from the virtual camera to the surface of the synthetic mesh. The use of paired synthetic images and ground-truth depth images is used to train the depth prediction model because monocular cameras do not directly indicate depth. Video sequences can be formed from the paired synthetic images and true depth images, as described in detail in relation to FIG. 7B, below.

In step 214, a virtual endoscopic dataset, of which virtual endoscopic dataset 720 of FIG. 7C is exemplary, is created. The virtual endoscopic dataset is a collection of video replication sequences. The various replication sequences are generated using different combinations of parameters, in order to replicate the various conditions that may be present when taking endoscopic imagery in airways.

In step 214, various parameters of the surface mesh may be altered, to provide unique surface geometry and texture for each video sequence. These parameters include camera properties (e.g. resolution, FOV, etc.), movement path, lighting, and special condition changes (e.g. effects from fluid, bubbles). In the real world setting, many of these parameters significantly vary the resulting image quality. A combination of parameters can be defined as a discrete property instance Pᵢ from the set: P={M(t), Cₚₐₜₕ(t), Cₚₐᵣₐₘₛ, L, E} where M(t) is a time dependent 3D surface mesh, Cₚₐₜₕ(t) is the camera path as a function of time, Cₚₐᵣₐₘₛ is the set of camera parameters, L is the set of lighting conditions, and E is the set of effects such as fluid, specular reflection, bubbles and other cofounding factors that degrade image quality.

Step 214 results in a dataset consisting of up to n video sequences of length m from each parameter instance Pᵢ, where n is the number of parameter combinations, and m is the number of frames in each video.

In step 216, the virtual endoscopic dataset is used to train the depth prediction model to predict or generate a depth image directly from an RGB image of an airway. Various GAN model architectures can be used to achieve this, such as the “Pix2Pix” GAN model. In an embodiment, a GAN architecture may be based on the extended Pix2Pix GAN. The GAN architecture may include an encoder-decoder architecture with skip connections between each layer. The GAN architecture may include a generator consisting of 8 encoder layers and 7 decoder layers, with the discriminator consisting of five layers. The GAN may be trained on using test images. In an embodiment, a GAN may be trained for 80 epochs using a batch size of 20 and a learning rate set to 0.0002. LeakyRelu may be used in the convolution layers for the generator, with a leaky relu alpha of 0.2. Relu activation functions may be used for deconvolution layers in the generator, with tan h being used for the output layer. An ADAM optimizer may be employed with β1=0.5. Batch normalization that may be included in a Pix2Pix GAN and may be omitted from the model.

### Airway Contouring Process

The depth classification system may generate contours of the airway using a trained depth prediction model. An implementation of the airway contouring process 200B is shown in FIG. 2.

In step 220 of the airway contouring process 200B, real-time monocular endoscopic video is received. Alternatively, in some embodiments, a recording of an endoscopic video may be utilized.

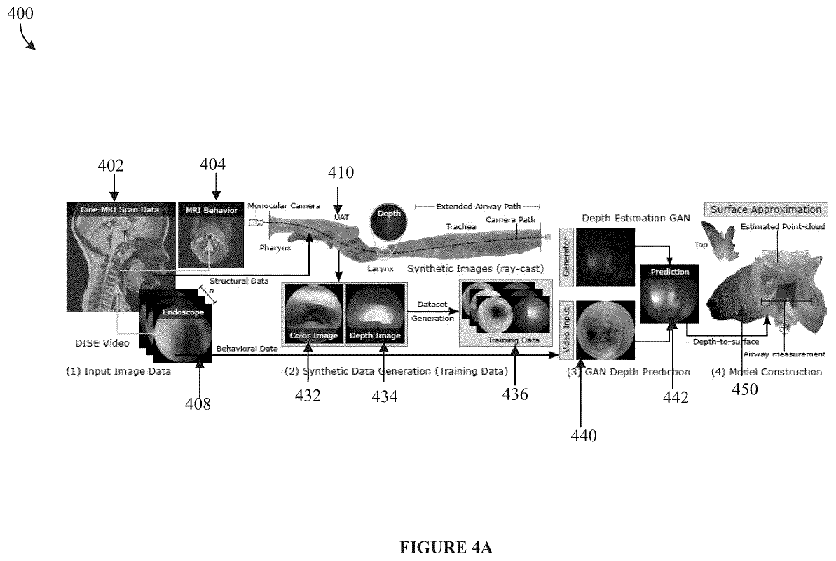

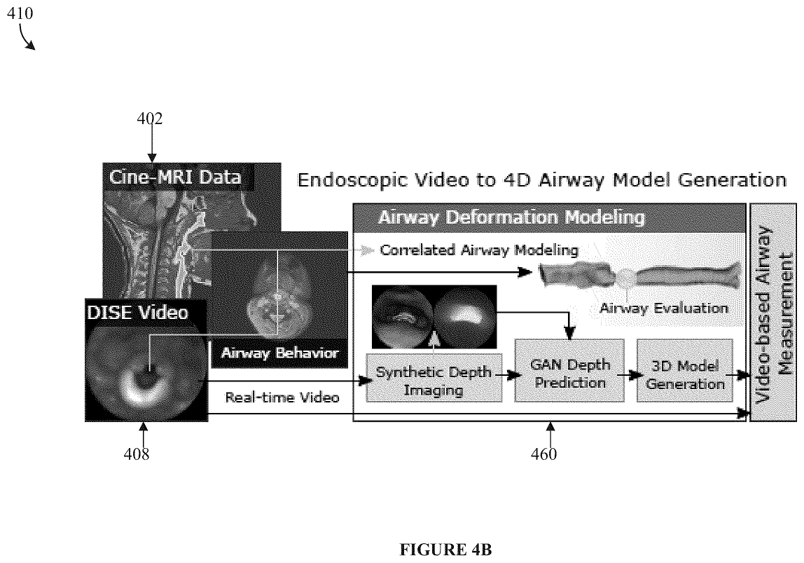

In step 222, a 3D point cloud surface is generated from a frame of the endoscopic video. The point cloud surface, of which point cloud surface 450 of FIG. 4A is exemplary, is a three-dimensional (3D) representation of the airway constructed based on the RGB frame and predicted depth data.

The generation of point cloud surface is based on the creation of surface patch estimates obtained from the endoscopic video. The point cloud surface is an approximation defining a discrete number of 3D point samples that lie on the observed surface. From the depth prediction model, the predicted depth image is used to translate pixel information from the endoscopic frame to 3D space. In this process, the predicted depth image may be insufficient on its own to compute the 3D coordinate of each pixel value. The intrinsics of the camera may be provided to generate the resulting point cloud surface. Each point in the point cloud surface is a function of the parameters and the predicted depth image:

P(x, y, z)=Cₚₐᵣₐₘₛ(D(i, j)).

Where P is the point in 3D space, Cₚₐᵣₐₘₛ are the parameters of the camera, and D(i, j) is the i, j pixel of the predicted depth image. In some embodiments, a surface normal (n) may be computed for each 3D point. In some embodiments, an RGB color value may be assigned to each corresponding 3D point.

In step 224, the dynamic airway model is generated. The dynamic airway model is a 4D (three spatial dimensions plus a time component) representation of the airway, that is, a 3D representation that shows changes over time. Deformation models may be used to construct a deformable model of the airway, such as template warping deformation models and real-time localized deformation models.

Template warp deformation modeling is based on an initial model of the airway that is constructed and then deformed by newly collected surface states local to a sub region of the model. This requires a two stage process where the initial model is generated from a series of static point-cloud surfaces along the entire UAT, and then the camera is moved to the point of interest to generate new point cloud surfaces that describe localized surface deformation. The camera can then be held at the point of interest to record changes within the airway.

In template warp deformation modeling, incremental global alignment (registration) of all point-clouds are generated. This process can be completed using any number of global point-cloud registration algorithms. The most popular method is Simultaneous Localization and Mapping (SLAM). This combines the discrete set of point-cloud surfaces into a densely sampled surface based on the overlap between point-clouds containing same sections of the airway. Some methods for global alignment can be used by estimating camera position from camera odometry data. The result of this process is a new 3D point-cloud which contains the points from all input scans, properly aligned to form the geometric structure of the recorded region of the airway. Various methods can be used to construct a triangulated approximation of the surface from the provided 3D point-cloud data. This includes Marching Cubes as well as Poisson-based surface reconstruction methods, and polygon-soup triangularization methods. After the initial surface model is generated the camera can be moved to a location already captured by the reconstruction. Feature correspondence is used to map new incoming video frames to identify the location within the reconstructed model to perform a warp. Following the same process as steps to generate a point-cloud surface of the local region, the constructed UAT surface model will be warped to the new point-cloud estimate. This will deform the surface of the model to the state of the point cloud surface based on matching key features from the new data to the existing location within the UAT model. The template warp deformation process results in a dynamic airway model, defined by discrete 3D surface states that contains the entire UAT as well as deformations to localized regions.

Real-time deformation modeling forgoes the initial creation of a static airway model and directly converts surfaces visible within the monocular camera at a single location to point cloud surfaces. This provides an immediate construction of the airway, but is limited to the airway visible to the camera. In real-time deformation modeling, various methods can be used to construct a triangulated approximation of the surface (the dynamic airway model) from the provided 3D point-cloud data (point cloud surfaces). This includes Marching Cubes as well as Poisson-based surface reconstruction methods, and polygon-soup triangularization methods.

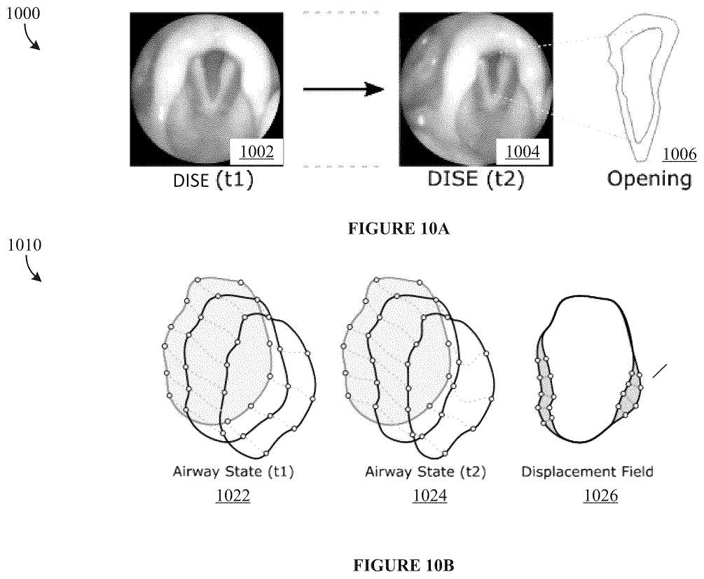

In step 226, contours of airway are generated. Exemplary airway contours 1002 and 1004 are shown in FIG. 10A. The airway contours represent the perimeter of the airway at a specific cross-section. The airway contours can be characterized by the intersection between a plane and the point cloud surface. The airway contours are quantitative measures obtained from the dynamic model to define the state of the airway opening and the degree of airway collapse.

Contour measurement is complicated by camera movement. This is because relative camera movement can impose an effective change in contour size based on distance to the airway opening. This is resolved through one of two methods: (1) stationary camera position may be maintained for the quantitative measurement or (2) camera odometry may be used to identify the motion path of the camera, accounting for the impact of the position of the camera on the apparent airway opening size.

In step 228, airway wall displacement is mapped. FIG. 10B illustrates an airway wall displacement mapping in an implementation, using a first contour 1022 at a first time, and a second contour 1024 at a second time. A displacement field 1026 is generated based on a comparison between the first contour 1022 and the second contour 1026. These displacement field may be displayed to a user, for example a doctor, to assist in classifying a condition of the airway.

Discrete topology states between frames (or every n-th frame) defines difference in airway wall topology (e.g., between successive contours) that can be represented through a displacement field. Regions within the airway are characterized by different potential displacements that naturally occur during breathing. This provides a natural fluctuation of a cyclical displacement field that describes natural airway wall deformation and movement. From the two given states, corresponding features can be identified to anchor displacements to a localized sub region within the airway. The resulting change in the reconstructed surface between both topologies is evaluated as a 3D displacement field. The displacement may also be characterized by deformation within the surface. This will introduce a nonlinear transformation between surface states. This is resolved by identifying key points within the deformation region to track between the two deformation states. The features are then used to identify the corresponding displacement field.

FIG. 3A illustrates axial views of for an exemplary cross-sectional airway measurement between two different breathing states from a Cine-MRI scan in an implementation. This axial view provides a quantitative description of the airway opening that can be used to evaluate airflow through behavioral analysis. This quantitative data is linked with DISE acquired video data and trained network model to obtain measurements directly from monocular videos of the airway.

FIG. 3B illustrates a process of virtualized airway modeling for augmenting network model training data through synthesized imaging in an implementation. Virtualized scan data is used to define structural airway models that are used for generating images that are correlated with ground-truth depth values provided through ray-casting algorithms. Surface mesh model 310 is a 3D construction of an airway from which synthetic RGB and depth data will be used for training a depth prediction model. The depth data is acquired relative to the position of virtual camera 320, the position of which corresponds to a measurement plane. Image 312 illustrates a synthetic RGB frame generated from the 3D surface mesh model. The synthetic RGB frame may be generated using surface mesh model 310. Image 314 represents a predicted depth frame corresponding to image 312.

FIG. 3C illustrates a functional set of training data used to build the correlative model between RGB image sequences from DISE video data with corresponding depth images in an implementation. Large datasets are formulated for different levels of estimation accuracy based on training data sources that range from synthetic data, 3D printed models, in vitro modeling, and DISE video. Images 332 are examples of synthetic RGB frames generated from a 3D surface mesh model, while images 334 are examples of predicted depth frames for the corresponding RGB images.

FIG. 4A illustrates operational overview 400 of a correlative data processing pipeline for the estimation of dynamic 4D airway surface models in an implementation. Described within this process is the collection of paired Cine-MRI scan data, DISE video sequences, and depth images assembled into datasets used to train the model for depth predictions and airway surface reconstruction. From the resulting airway model, dynamic structural changes and deformations can be quantitative evaluated through model space coordinates defined by the paired Cine-MRI data. Surface mesh model 410 is generated from Cine-MRI data 402. Paired with Cine-MRI data is actual endoscopic imagery data 408. Image 432 illustrates a synthetic RGB image produced from surface mesh model 310 and actual endoscopic imagery 408. Image 434 illustrates a depth comprising predicted depth data derived from surface mesh model 410. Multiple sets of paired images (of which images 432 and 434 are typical) comprise training dataset 436. Training dataset 346 is used to train a depth prediction model, which is a machine-learning-based model which predicts depth data from RGB image data. Training datasets may also comprise a parametrization by which parameters can be varied to broaden the scope of the training. These parameters may include camera exposure data, lighting data, airway surface characteristics, image artifacts, and other confounding variables. Image 442 illustrates the predicted depth data for an inputted video image 440. A set of RGB images with corresponding predicted depth data images are used to generate point-cloud surface approximation 450. Point-cloud surface approximation 450 may be subsequently used to generate a dynamic, i.e., 4D, model of the airway or to classify airway behavior, airway obstructions, or to quantify airway characteristics.

FIG. 4B illustrates an airway evaluation pipeline leading from DISE video data (e.g., endoscopic imagery data 408) paired with Cine-MRI scan data (for example, Cine-MRI data 402) to produce 4D airway models in an implementation. This introduces a diagnostic system that can be used to provide conditional evaluation through video-based airway measurements and behavioral analysis. Airway deformation model 460 comprises generating a 3D model of the airway which is used for airway evaluation, classification, diagnostic procedures, etc. Airway deformation model 460 also comprises generating a 3D model, wherein predicted depth values are generated from the real-time endoscopic imagery using a depth prediction model trained from data sets such as training dataset 436. From airway deformation model 460, a video-based system for classifying and quantifying the airway or specific locations within the airway can be generated.

FIG. 5A illustrates synthetic video sequence data and paired ground-truth depth data used to train and validate the depth prediction model used by a depth prediction module in an implementation. Images 502 illustrate synthetic RGB images generated from a 3D surface mesh model, e.g. surface mesh 310 of FIG. 3B, which was in turn generated from Cine-MRI data. Ground-truth depth images 504, corresponding to images 502, illustrate depths quantified from the 3D surface mesh model. Images 502 are then run through the depth prediction model to generate predicted depth frames 506. Comparing predicted depth frames 506 to ground-truth images 504 yields difference maps 508. The difference maps or quantifications can be used by self-supervising machine learning methods such as GAN to validate or train the model.

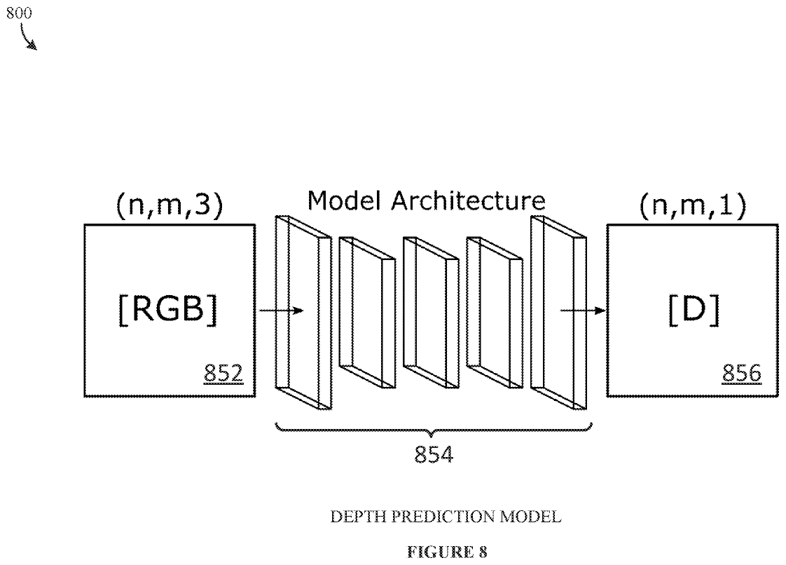

FIG. 5B illustrates a real-world implementation of the depth prediction model to estimate depths on low quality DISE video data sequences. In vivo image data 512 are run through a depth prediction model, of which depth prediction model 800 of FIG. 8 is representative, to generate predicted depth frames 516, in other words, predicted depth frames 516 illustrate output from a depth prediction model operating on image data 512. Predicted depth frames 516 can be used to generate a point-cloud surface model, 3D model, 4D dynamic model, or other model of the airway.

Similar to FIG. 5A, FIG. 5C illustrates depth estimations on synthetic video sequence data in an implementation. In the training process, the model generates synthetic predicted depth image 506 for each of synthetic RGB images 502. The synthetic predicted depth images 506 are then compared to the true depth images 504 to train and validate the model. The model uses this comparison to improve model accuracy. Images 560 comprise images of contours generated from a point-cloud surface model based on RGB images 501 and depth frame 506. Comparing the open airway contour with the restricted airway contour allows obstructions to be classified for diagnosis and treatment.

### Classification Process

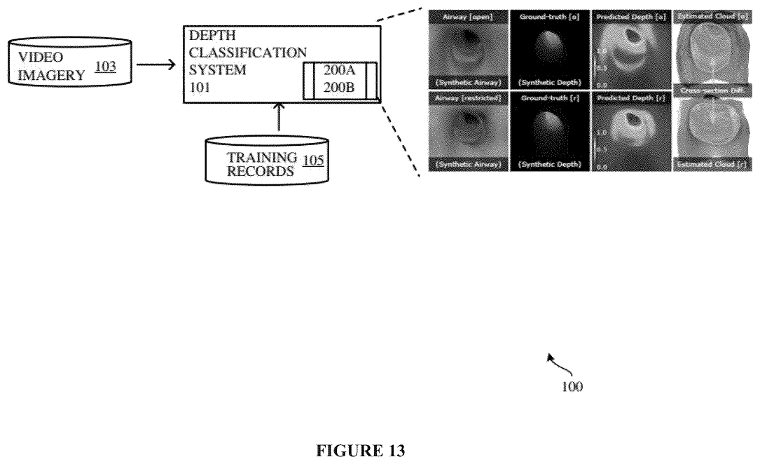

In some embodiments, the depth classification system includes a depth classification model to determine whether there are obstructions in the airway and classify the level of obstruction. An exemplary classification model is shown in FIG. 13.

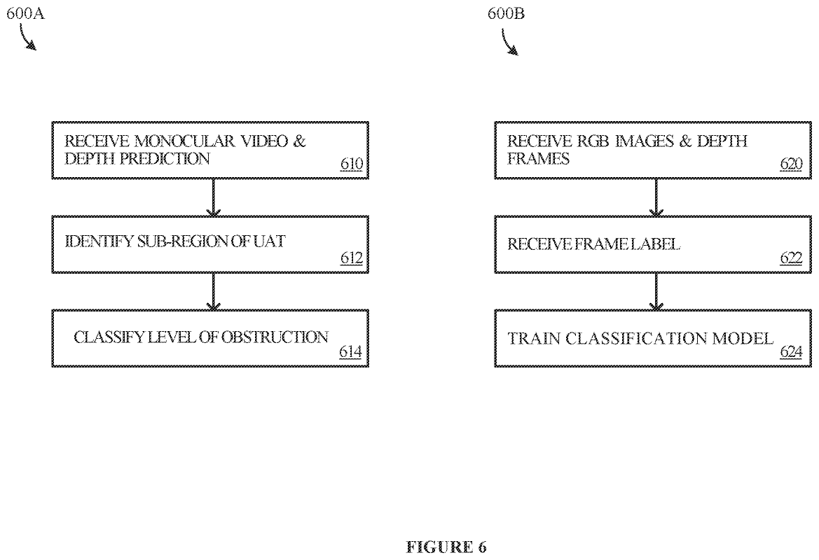

The classification model classifies airways using classification process 600A shown in FIG. 6. In step 610, the classification model receives the paired endoscopic RGB images and endoscopic predicted depth images generated from the monocular video imagery of the airway. In step 612, the classification model identifies a subregion of the UAT based on the endoscopic RGB images and endoscopic predicted depth images received. The subregion is specific region of the airway that is shown in the endoscopic video (e.g. Velum, Oropharynx, Tongue-base, Epiglottis). In step 614, the classification model determines an airway classification based on the subregion identified and the paired endoscopic RGB images and predicted depth images. In some embodiments, the airway classification indicates a level of obstruction in the subregion of the airway. The classification model may output a score from 0 to 3, where 0 indicates the lowest level of obstruction, and 3 indicates the highest level of obstruction. Alternatively, any suitable grading scale may be used.

In some embodiments, the classification model may additionally use a point cloud surface, airway contours with a displacement field, or any combination of the above, to determine the subregion and airway classification.

### Classification Model Training Process

A classification model is trained using a classification model training process 600B as shown in FIG. 6.

In step 620 of the classification model training process 600B, according to some embodiments, the classification model receives synthetic RGB images and synthetic predicted depth images. In other embodiments the model receives the endoscopic RGB images from the actual monocular camera, along with the paired endoscopic predicted depth images from the monocular camera. In step 622, the classification model receives the frame label for the endoscopic images. The frame label may correspond to the specific region of the airway that is shown (e.g. Velum, Oropharynx, Tongue-base, Epiglottis).

In step 624, the classification model is trained to correctly identify the subregion. In some embodiments, the classification model predicts a local subregion and compares to predicted subregion to the frame label received in step 622. The model is then updated for accuracy based on this comparison. The classification model may also be trained to identify the level of obstruction in the airway. Specifically, the classification model may generate a predicted level of obstruction, and compare it to a received actual level of obstruction. As discussed above, the level of obstruction may be indicated by a score from 0 to 3, where 0 indicates the lowest level of obstruction and 3 indicates the highest level of obstruction.

FIG. 7A illustrates representations of a DISE Frame and a synthetic frame paired with a true depth frame in an implementation. DISE provides single RGB frames, e.g., image 702. The synthetic image generation process produces synthetic endoscopic image RGB 704 as well as associated depth image 706, therefore the output of the synthetic image generation process is image pair RGB-D, i.e., images 704 and 706. Images 308, 312, and 314 are examples of images 702, 704, and 706, respectively.

FIG. 7B illustrates video sequence 710 formed from paired frames generated by the synthetic image generation process in an implementation. A synthetic image and corresponding depth frame may be generated for multiple frames to create video replication sequence 710. Video replication sequence 710 is a series of frames of synthetic images 704 (replicating an endoscopic video), with a corresponding true depth image 706 for each frame. Generation of the synthetic RGB-D image pairs (including, for example, image pair 704 and 706) replicating the DISE video sequences may depend on the selected virtual 3D airway model and camera movements or paths. Thus, several unique sequences can be generated.

FIG. 7C illustrates a representation of training dataset 720 including paired video sequences 710, 712, and so on, according to a parameter set in an implementation. “n” videos of length “m” are generated based on the various of parameters defining within each parameter instance “Pᵢ.” This generates unique video sequences, each with image variance. Parameters can include conditions which can be varied to adjust image quality, such as camera properties (e.g., exposure, resolution, field of view or FOV), camera movement path, lighting, and special condition changes, for example, effects from fluid, bubbles, specular reflection and other confounding factors that degrade image quality.

Training dataset 720 is useful for model training because monocular endoscopic imaging cannot directly provide depth data required for 3D reconstructions. The depth prediction model estimates a depth value for each pixel within the RGB image. Self-supervised methods in machine learning have been developed to perform this mapping. This can include “Pix2Pix” image translation architecture built as a Generative Adversarial Network (GAN). The purpose of the GAN is to transform one image into another based on training. The presently disclosed method is used to predict depth images directly from RGB endoscopic images. To do this, the transformation function within the model architecture will define a RGB-to-Depth mapping. Training data for this transformation includes the synthetic images and the corresponding true depth image. This data is generated through the processes described herein. Multiple data sequences are generated by varying parameters to replicate real-world DISE videos. These parameters include camera properties, camera movement, lighting, and complex simulated effects that may include liquid within the airway, bubbles, and tissue characteristics. Video sequences such as video sequences 710, 712, and so on are generated for each combination of these parameters.

FIG. 8 illustrates a depth prediction model architecture in an implementation. The depth prediction model, which may be employed by depth prediction module 130 in an implementation, generates depth information of the airway corresponding to video imagery data 853. In an implementation, the depth prediction model receives video imagery data 853 in the form of RGB (Red, Green, Blue) images and outputs a predicted depth image. The RGB images may be, in an implementation, an endoscopic RGB frame extracted from the video imagery data. In the training process of the depth prediction model, the RGB images may be synthetic images of an airway. The depth prediction model may be based on deep learning or machine learning processes, such as GAN, to generate predicted depth information from video imagery data 853. The depth prediction model used by a depth prediction module, such as depth prediction module 130, is trained on datasets comprising synthetic DISE imagery data with synthetic depth data derived from Cine-MRI data, but may also be trained on real DISE imagery data with derived depth data derived from corresponding Cine-MRI data.

FIG. 9 illustrates airway contours extracted from an MRI scan in an implementation. In an implementation, a surface mesh is created from a sequence of Cine-MRI data images. Referring to Cine-MRI image 902 as an example of the process applied to a single image, airway contour approximation 904 is extracted from Cine-MRI image 902. Airway contour approximation 904 represents a contour of the airway at a particular location in the airway. Various methods can be used to extract airway contour approximation 904, for example, the extraction may be accomplished using image edge detection, gradient analysis, or Cine-MRI segmentation through U-Net CNN architectures.

Multiple airway contour approximations at incremental axial locations may be stacked together to create axial stack 906 of airway contours, which provides a definition of the airway walls. To create axial stack 906, contour ring normals î are estimated using the equation \hat{n}=(ŷ×\hat{x}); where ŷ=up, \hat{x}=along contour. Axial stack 906 can thus give the approximate form and surface direction of the airway walls obtained from the sequence of Cine-MRI data. Axial stack 906 with the estimated normals î form an approximation of visible UAT wall tissue.

Axial stack 906 is used to generate the surface mesh, such as surface mesh 310 of FIG. 3B. This process may be performed for several Cine-MRI scans, leading to multiple surface meshes, improving the quality and dataset size that can be generated for training and validation of the depth prediction model. Real endoscopic imagery may also be used to create the surface mesh, in addition to the Cine-MRI data. The real endoscopic imagery is used for surface information (e.g. color and texture information). The surface mesh may be stored as a triangulated mesh that describes the surface topology of the airway as defined by Cine-MRI data. Various methods may be used to convert axial stack 906 into a surface mesh. These methods include Marching Cubes, Poisson-based surface reconstruction, or triangulation methods to create a continuous triangulated mesh based on the provided input. The surface mesh may be defined as a set of nodes, triangles, edges, and other basic primitives that form the surface mesh.

With axial stack 906 generated, a 3D surface mesh representation of the UAT is created. The surface mesh is used to create synthetic images, such as synthetic image 312 of FIG. 3B, imitating endoscopic imagery. To clean and reduce the complexity of the surface mesh, in some embodiments this may be accomplished by collapsing small triangles, resolving distorted regions within the mesh, and removing disconnected components.

The synthetic images replicate the visual fidelity of real-world DISE video frames through synthetic imaging techniques. In some embodiments, ray-tracing and other graphics techniques are used. The surface mesh provides the geometry that defines the structure of the airway to be rendered in the synthetic image creation process. The synthetic image is paired with a depth frame “D” (n, m, 1), where each pixel of the n by m pixel array of the depth frame comprises a distance between the camera or measurement plane and airway wall surface for each DISE video frame (1=single channel grayscale).

The depth frames, of which image 314 of FIG. 3B is exemplary, represent distances between a virtual camera and the surface mesh. Each pixel in the depth frame may represent a distance between a virtual camera and the surface mesh, such as virtual camera 320 and surface mesh 310 of FIG. 3B. The depth frames may be generated using positional data from the surface mesh, as well as the camera properties, including the location of the virtual camera within the surface mesh. The true depth frames may be represented in grayscale. The process may produce a video sequence of the grayscale true depth images.

The generation of synthetic RGB images and depth frames may include generating or obtaining surface textures of the airway, loading airway surface states of the surface mesh (each stored as an individual snapshot of topology), replicating of camera properties (e.g. camera FOV, distortion) replicating camera movements, and replicating lighting conditions (e.g. lighting model, intensity, impact on glare and specular reflections). Finally, ray-tracing may be performed for each frame, replicating the visuals presented within monocular endoscopic cameras. The ray-tracing process provides both the synthetic image and the true depth image.

FIG. 10A illustrates real DISE imagery of a UAT of a patient. A quantitative measure to obtain from a dynamic model of an airway is the state of the airway opening or the degree of airway collapse. FIG. 10A illustrates a first contour 1002 of an airway position at a first time, and a second contour 1004 of the airway position at a second time, the contours having been generated from a dynamic model of the airway. The contours 1002 and 1004 may be overlayed on images from the monocular camera. Image 1006 illustrates a comparison of the airway openings between contours 1002 and 1004. Images 1002 and 1004 may then be displayed to a user, for example a doctor, to assist in classifying a condition of the airway.

Once a point cloud surface representation of an airway has been generated from RGB image data and depth data, various quantifications and classifications can be performed. FIG. 10B illustrates an airway wall displacement mapping in an implementation, using a first contour 1022 at a first time, and a second contour 1024 at a second time. A displacement field 1026 is generated based on a comparison between the first contour 1022 and the second contour 1024. Displacement field 1026 may be displayed to a user, for example a doctor, to assist in classifying a condition of the airway.

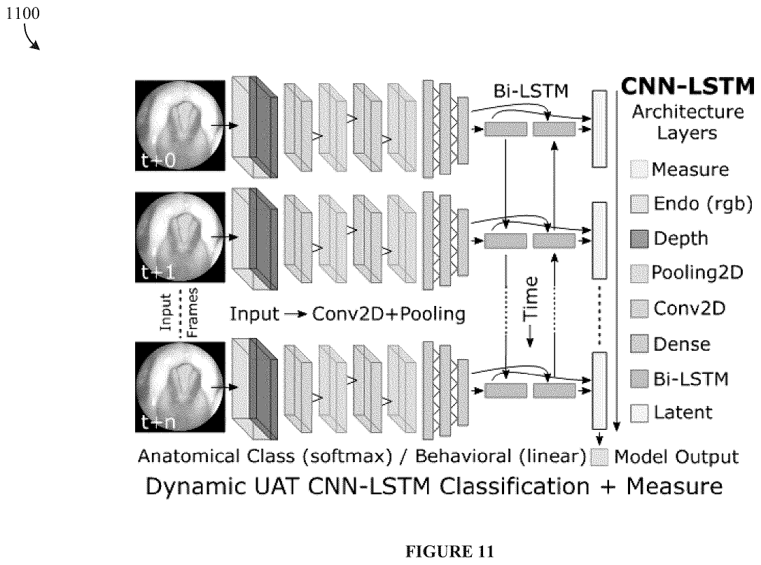

In some implementations, subsequent to depth prediction and generation of a point-cloud surface model, a classification model can be used to determine whether there are obstructions in the airway and to classify the level of obstruction. An exemplary classification model is shown in FIG. 11.

Classification model 1100 of FIG. 11 classifies airways using a classification process for which process 600A shown in FIG. 6 is exemplary. The classification model receives the paired endoscopic RGB images and endoscopic predicted depth images generated from the monocular video imagery of the airway. The classification model identifies a subregion of the UAT based on the endoscopic RGB images and endoscopic predicted depth images received. The subregion is specific region of the airway that is shown in the endoscopic video (e.g. Velum, Oropharynx, Tongue-base, Epiglottis). The classification model also determines an airway classification based on the subregion identified and the paired endoscopic RGB images and predicted depth images. In some implementations, the airway classification indicates a level of obstruction in the subregion of the airway. The classification model may output a score from 0 to 3, where 0 indicates the lowest level of obstruction, and 3 indicates the highest level of obstruction. Alternatively, any suitable grading scale may be used.

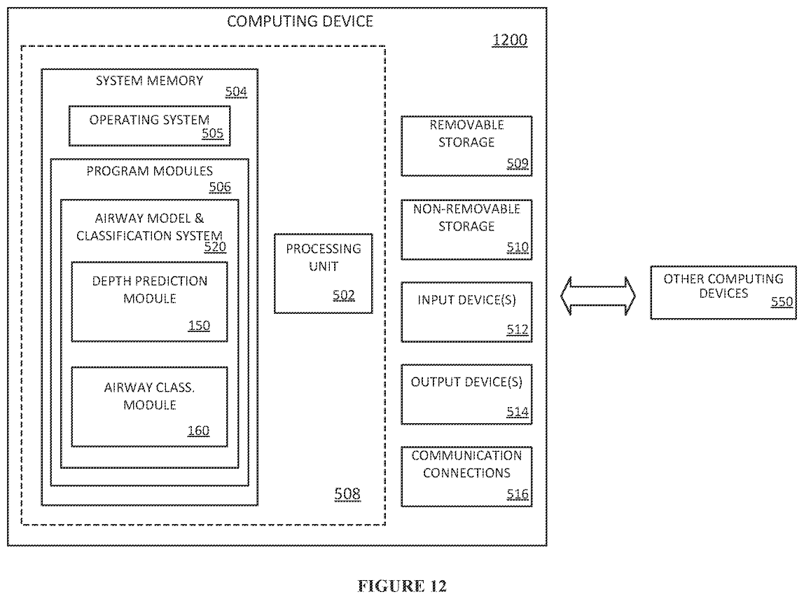

FIG. 12 is a block diagram illustrating physical components (e.g., hardware) of a computing device 1200 with which aspects of the disclosure may be practiced. The computing device components described below may have computer executable instructions for assessing airway deformation. In a basic configuration, the computing device 1200 may include at least one processing unit 502 and a system memory 504. Depending on the configuration and type of computing device, the system memory 504 may comprise, but is not limited to, volatile storage (e.g., random access memory), non-volatile storage (e.g., read-only memory), flash memory, or any combination of such memories. The system memory 504 may include an operating system 505 suitable for running one or more airway deformation processing programs. The operating system 505, for example, may be suitable for controlling the operation of the computing device 1200. Furthermore, embodiments of the disclosure may be practiced in conjunction with a graphics library, other operating systems, or any other application program and is not limited to any particular application or system. This basic configuration is illustrated in FIG. 12 by those components within a dashed line 508. Computing device 1200 may have additional features or functionality. For example, computing device 1200 may also include additional data storage devices (removable and/or non-removable) such as, for example, magnetic disks, optical disks, or tape. Such additional storage is illustrated in FIG. 12 by a removable storage device 509 and a non-removable storage device 510.

As stated above, a number of program modules and data files may be stored in the system memory 504. While executing on the processing unit 502, the program modules 506 may perform processes including, but not limited to, the aspects, as described herein. Program modules 506 may include processes 200A, 200B, 600A, and 600B, which may be deployed as described in FIGS. 1-11 and 13-17 herein.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. For example, embodiments of the disclosure may be practiced via a system-on-a-chip (SOC) where each or many of the components illustrated in FIG. 12 may be integrated onto a single integrated circuit. Such an SOC device may include one or more processing units, graphics units, communications units, system virtualization units and various application functionality all of which are integrated (or “burned”) onto the chip substrate as a single integrated circuit. When operating via an SOC, the functionality, described herein, with respect to the capability of client to switch protocols may be operated via application-specific logic integrated with other components of the computing device 1200 on the single integrated circuit (chip). Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general-purpose computer or in any other circuits or systems.

Computing device 1200 may also have one or more input device(s) 512, which receives the monocular video data and camera parameters. The one or more input device(s) may also include a keyboard, a mouse, a pen, a sound or voice input device, a touch or swipe input device, a gesture or visual input device, etc. The output device(s) 514 such as a display, speakers, a printer, etc. may also be included. The aforementioned devices are examples and others may be used. The computing device 1200 may include one or more communication connections 516 allowing communications with other computing devices 515. Examples of suitable communication connections 516 include, but are not limited to, radio frequency (RF) transmitter, receiver, and/or transceiver circuitry; universal serial bus (USB), parallel, and/or serial ports.

The term computer readable media as used herein may include computer storage media. Computer storage media may include volatile and nonvolatile, removable, and non-removable media implemented in any method or technology for storage of information, such as computer readable instructions, data structures, or program modules. The system memory 504, the removable storage device 509, and the non-removable storage device 510 are all computer storage media examples (e.g., memory storage). Computer storage media may include RAM, ROM, electrically erasable read-only memory (EEROM), flash memory or other memory technology, CD-ROM, digital versatile disks (DVD) or other optical storage, magnetic cassettes, magnetic tape, magnetic disk storage or other magnetic storage devices, or any other article of manufacture which can be used to store information, and which can be accessed by the computing device 1200. Any such computer storage media may be part of the computing device 1200. Computer readable media does not include a carrier wave or other propagated or modulated data signal. Computer readable storage device does not include a carrier wave or other propagated or modulated data signal.

Communication media may be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media.

Some embodiments include a method for model-based analysis of dynamic airway evaluation comprising: a combination of multiple imaging modalities that are integrated into a multi-modal data aggregate that depict airway structures as obtained from various forms of scanning technologies including Cine-MRI, monocular endoscopic video, and CT scans and a systematic method for generating structural and behavioral models of airway dynamics and surface deformations directly from video data a method for replicating airway structures through virtual and 3D printed models used for training models that convert image data to structural information and the generation of airway deformation behaviors through depth estimations that provide descriptors for pulmonary functionality and conditions.

Some embodiments include a system as shown and described herein, and equivalents thereof comprising: Cine-MRI, CT, and other forms of medical scans with monocular video data of the airways through the collection and analysis of airway deformations using image data and the data from these individual scans are integrated into a prediction and measurement system that obtains quantitative evaluations of cross-sectional area, perimeter, depth, curvature, and surface estimates that comprise the system for extracting surface dynamics.

Some embodiments include method for integrating multiple scanning and imaging modalities (Cine-MRI, CT, monocular video, etc.) that independently describe pulmonary functionality comprising: a method for correlating airway features from one modality to another through GAN model predictions, and an integration procedure for producing quantitative physical measurements and units from one modality to another generation and integrated blend of synthesized data from multiple imaging modalities (Cine-MRI, CT, video, etc.) into training sets, and features identified within one imaging modality are correlated into a behavioral model describing dynamic surface behaviors of the airways.

Some embodiments include a method of training models for the measurement and classification of airway passageway deformations and behavioral analysis, the method comprising: generating synthetic monocular video sequences of the airway walls; generating depth data of virtual airway passageway model; training GAN based models on synthetic monocular video and the depth data for airway deformation reconstruction and modeling; obtaining video feed and integrating advance scan data to from multi-modal data aggregates for airway modeling; estimating depth maps for the video feed based on the trained GAN; and constructing point-cloud and surface approximations of airway deformations, constriction, and obstruction sites.

Some embodiments include a method for obtaining pulmonary functionality metrics from the model and cross-referencing data between training datasets to provide guided analysis of deformation behaviors, airway restrictions, and obstruction sites.

Some embodiments include a method of classifying airway passageway deformation comprising: identifying surface deformation characteristics from within a video feed; linking characteristics of video feed with airway flow data; and classifying airway passageway deformations.

Some embodiments include a method of predicting airway passageway deformation comprising: obtaining live video feed; identifying characteristics of the live video feed; correlate the characteristics of the live video feed with trained GAN; and generate surface approximation comprising measurements of the airway passageway deformation.

Aspects of the present disclosure, for example, are described above with reference to block diagrams and/or operational illustrations of methods, systems, and computer program products according to aspects of the disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

The description and illustration of one or more aspects provided in this application are not intended to limit or restrict the scope of the disclosure as claimed in any way. The aspects, examples, and details provided in this application are considered sufficient to convey possession and enable others to make and use the best mode of claimed disclosure. The claimed disclosure should not be construed as being limited to any aspect, example, or detail provided in this application. Regardless of whether shown and described in combination or separately, the various features (both structural and methodological) are intended to be selectively included or omitted to produce an embodiment with a particular set of features. Having been provided with the description and illustration of the present disclosure, one skilled in the art may envision variations, modifications, and alternate aspects falling within the spirit of the broader aspects of the general inventive concept embodied in this application that do not depart from the broader scope of the claimed disclosure.

The various embodiments described above are provided by way of illustration only and should not be construed to limit the claims attached hereto. Those skilled in the art will readily recognize various modifications and changes that may be made without following the example embodiments and applications illustrated and described herein, and without departing from the true spirit and scope of the following claims.

FIG. 13 illustrates an operational architecture 100 in an implementation of enhanced classification technology for assessing airway deformation. Operational architecture 100 includes depth classification system 101, video imagery 103, and training records 105. Depth classification system 101 employs training process 200A and classification process 200B. Video imagery 103 may include DISE video, monocular video sequences, and the like. Training records 105 may include synthetic models, 3D printed models, fluid flow evaluations using computational fluid dynamics, general fluid flow data, etc. Training records 105 may also include endoscopic video procedures linked to Cine MRI, 3D reconstruction of airway, airway surface model datasets (e.g., GAN model datasets), etc.

Depth classification system 101 generates quantitative data through ML techniques that analyze characteristics of the content of video imagery 103 with the content of training records 105 to provide a 4D model (e.g., a 3D time-series model). The 4D model may encode the surfaces, deformations, and/or overall behavioral description of the airway (e.g., as a set of surface states over time). The 4D model may be implemented for use in surgical interventions and/or decision making. The 4D model may also be implemented to provide structural and behavioral information of the content of video imagery 103 for post DISE analysis. For example, a depth-to-surface estimate provided through the use of GAN may be used to generate estimates of airway surface structures, provide direct feedback of 3D surfaces of airways (e.g., during DISE procedures), to model airway deformation characteristics, to measure dynamic behaviors of the airway over time, and the like.

Depth classification system 101 enables quantitative evaluation of airway behaviors using DISE video supplemented through Cine MRI data to obtain accurate measurements of airway restrictions and degree of airway collapse at obstruction sites (e.g., obstruction sites common to OSA and other related Otolaryngic and Ear, Nose and Throat conditions).

For example, depth classification system 101 may utilize 3D printed and/or molded silicone models to generate digital, anatomical models of airways. The anatomical models of airways may be created by loading CT and/or Cine MRI scan data (e.g., DICOM files) into a 3D slicer. Similarly, airways may be constructed using CT scan data to perform evaluations on chemical traversal times. Depth classification system 101 may use CT and/or Cine MRI imaging to obtain quantitative measurement of behavioral changes in an airway, including conditions like OSA. Cine MRI is capable of recording all levels of an airway (e.g., the upper airway) simultaneously and provides an objective measurement of the internal structure and behavior of an airway.

Depth classification system 101 may use GAN techniques to achieve endoscopic anatomical 3D reconstruction of an airway. Depth classification system 101 may implement GAN to predict depth from monocular images and train models for determining surface estimates of airway deformations, including wall movement and opening measurements to further determine obstruction severity. Depth classification system 101 may use CheMRI in conjunction with the predicted model to correlate and adjust any outlying dynamic data.

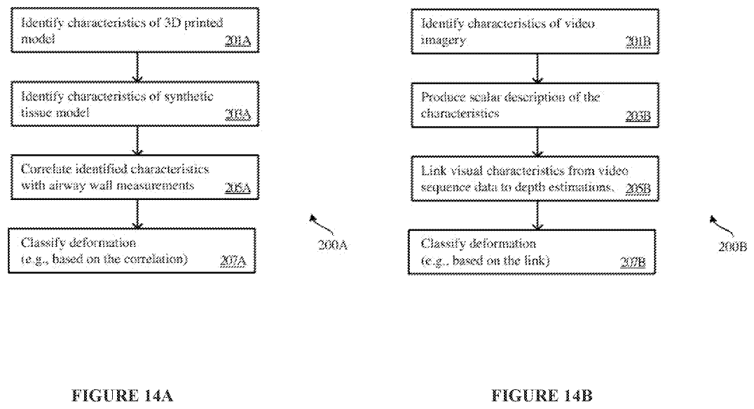

Depth classification system 101 may use real-time video inputs while employing training process 200A, as illustrated in FIG. 14A, and classification process 200B, as illustrated in FIG. 14B.

Depth classification system 101 may incorporate one or more different types of image data. The different types of image data may include CT scans (e.g., for structural modeling), Cine MRI (e.g., for observing airway deformation patterns), endoscopic video, and the like. In an embodiment, depth classification system 101 integrates the one or more different types of imaging to form dynamic 4D models of airway behavior. The 4D models may be defined as surface states generated in 3D models over time that can be used for quantitative evaluation of airways. The 4D model may operate directly from DISE monocular video data. Utilizing the content of training records 105 (e.g., a trained depth-image generating GAN), depth classification system 101 predicts depth in endoscopic images and/or reconstructs the internal dynamic structure of observable airways.

Depth classification system 101 may reconstruct the visible geometry of the airway. Depth classification system 101 may measure various opening and classify behavioral characteristics of the airway based on one or more generated 3D models. Depth classification system 101 may employ synthesized ray-traced imaging data to generate color (e.g., endoscopic) and/or depth images for training the GAN (e.g., to correlate distance estimates between an endoscopic camera and discrete locations on observable surfaces). This provides a parallel to the real images obtained (e.g., with a monocular camera) that may be used by the trained GAN to estimate depth in new and/or real-time images. Depth classification system 101 may further use the trained model to predict depth images that can be converted to point-clouds that approximate the walls of the airway during the DISE procedure.

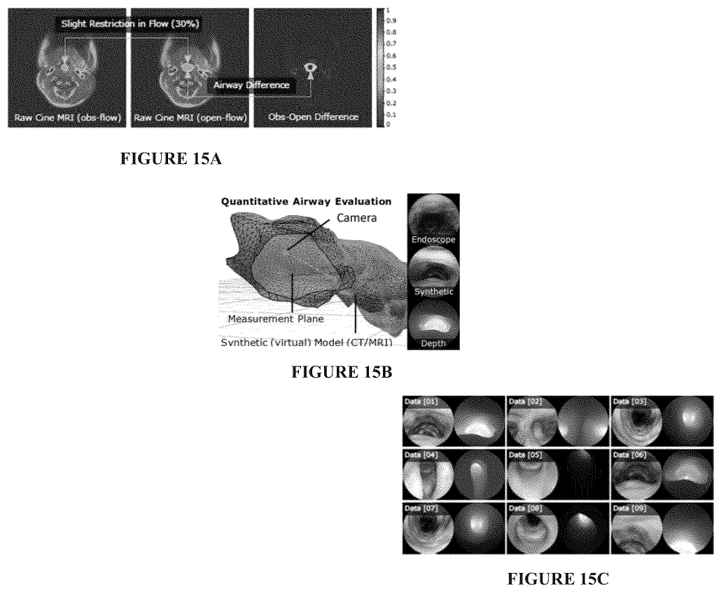

In an embodiment, depth classification system 101 may perform the steps of receiving DISE and Cine MRI data; generating synthetic monocular depth data of virtual airway models (e.g., to utilize as training input for the GAN); training the GAN; estimating depth maps for each frame (e.g., based on the GAN; and constructing surface models (e.g., point-clouds, surface states, etc.) that define observable dynamic conditions. FIG. 15A is illustrative of a Cine MRI scan obtained by depth classification system 101. The Cine MRI scan illustrates the change in airway diameter demonstrating the difference between restricted (e.g., obstructed) and open airway flow. The cross-section area is highlighted in the different image frames.

Inter-modality modeling may be generated by depth classification system 101, for example, by linking monocular video data with dynamic airway behaviors (e.g., airway behaviors captured in synchronized Cine MRI scans). The integration of these systems of imaging generates dynamic deformable models of recorded airway behavior. Cine MRI may also provide a quantitative baseline for establishing physical units of the depth images generated during surface reconstruction phases of modeling.

### Synthetic DISE Data Generation

To train one or more models (e.g., a model located in training records 105) on the relationship between endoscopic and depth images for airway surface estimation, depth images may be generated using a synthetic model (e.g., an anatomic model of an airway, portion of a GI tract, etc.). Synthetic models may represent a combination of 3D scan models that provide accurate structural and/or behavioral data that may be combined with endoscopic images to create virtual models. Cross-sectional areas of the virtual model may be directly compared to cross-sectional areas of predicted airway models (e.g., to determine accuracy of behavior models). Behavioral models may be further validated using a real camera and 3D printed model of an airway to simulate the DISE process on the 3D printed model (e.g., having known dimensions). Measurements obtained by manual airway probing (e.g., performed during DISE evaluations) may also be integrated into training the GAN (e.g., to evaluate cross-sectional measurements obtained from behavioral models).

In an embodiment, depth classification system 101 employs a 3D surface model that represents the anatomy of a patient's airway to create the monocular video feed, as well as the depth images for each frame. The monocular video feed may be created using a camera (e.g., a virtual camera) to traverse the 3D surface model of the airway. The movement of the camera may be controlled by a Bezier curve path. The camera may record a ray-traced image for each step illustrated in FIG. 15B. Color and depth images may be generated through batched parallel ray-tracing with a Phong lighting model using diffuse and specular maps. A light source may be attached to the camera and may have a linear attenuation (1/d). Randomized noise may be added to both color and depth samples. FIG. 15C illustrates training input pairs of color video frames and corresponding depth images used to train a GAN model located in training records 105.

By combining predicted depth image data with the intrinsic characteristics of a camera, depth classification system 101 may reconstruct an approximation of the observed airway surfaces as a dense point-cloud. Using the point-cloud, depth classification system 101 may generate 3D surface model comprising structural relationships that can be measured. For example, a cross-sectional region of interest may be selected, and the surface model may be queried to obtain a contour of the region of interest from which a direct measurement may be generated and/or otherwise obtained.

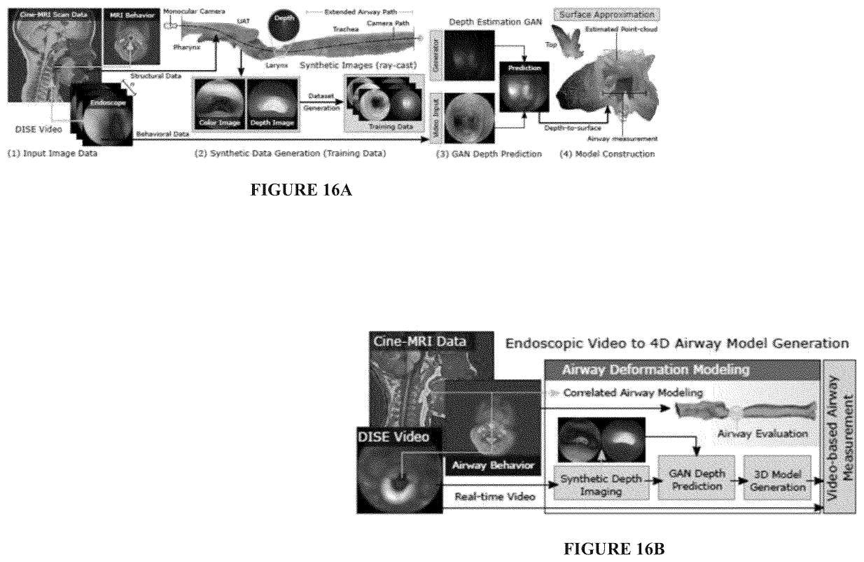

FIG. 16A illustrates an operational overview of the correlative data processing process for estimating 4D (i.e., dynamic) airway surface models. Described within this process is the collection of paired Cine-MRI scan data, DISE video sequences, and depth images assembled into datasets used to train the model for depth predictions and airway surface reconstruction. From the resulting airway model, dynamic structural changes and deformations can be quantitative evaluated through model space coordinates defined by the paired Cine-MRI data.

### GAN Architecture and Training

A monocular frame may be an input used to train a GAN model, which may output a corresponding depth prediction image (e.g., based on the training data). Depth prediction images may be used to reconstruct airway surface structures. Due to complexity in tracking of surface features, handling specular reflections due to fluids, and modeling complex structures, this method reduces the problem space of ill-conditioned monocular frames by inferring structural relationships between color and depth data within the trained GAN model.

In an embodiment, a GAN architecture may be based on the extended Pix2Pix GAN. The GAN architecture may include an encoder-decoder architecture with skip connections between each layer. The GAN architecture may include a generator consisting of 8 encoder layers and 7 decoder layers, with the discriminator consisting of five layers. The GAN may be trained on using test images. In an embodiment, a GAN may be trained for 80 epochs using a batch size of 20 and a learning rate set to 0.0002. LeakyRelu may be used in the convolution layers for the generator, with a leaky relu alpha of 0.2. Relu activation functions may be used for deconvolution layers in the generator, with tan h being used for the output layer. An ADAM optimizer may be employed with β1=0.5. Batch normalization that may be included in a Pix2Pix GAN and may be omitted from the model. FIG. 16A. illustrates an operational scenario in an implementation of training a GAN to predict depth on synthetic data. The difference is computed as the absolute difference between the ground-truth and predicted images.

FIG. 16B illustrates an airway evaluation process starting with DISE video data paired with Cine-MRI scan data to produce dynamic or 4D airway models. This introduces a diagnostic system that can be used to provide conditional evaluation through video-based airway measurements and behavioral analysis.

### Behavioral Reconstruction and Modeling

DISE video provides an effective method for identifying behavioral traits associated with obstructed breathing. High frame rates of endoscopic cameras that capture behavioral traits (e.g., of the respiratory cycle) may be leveraged to reconstruct observed surfaces and predict depth measurements. By using the trained GAN network, depth classification system 101 generates depth images for every frame collected from the monocular video. The depth images may be used in the reconstruction of a surface estimate that can be used to perform various measurements of restriction and flow reduction (e.g., relating to airflow and/or other metrics in DISE procedures). For the synthetic training data, surface deformations may be imposed on the reference model, replicating airway restrictions. The surface deformations may generate varying depth images and subsequent surface meshes that may be evaluated to identify a reduction in flow through cross-sectional regions of the observed airway.

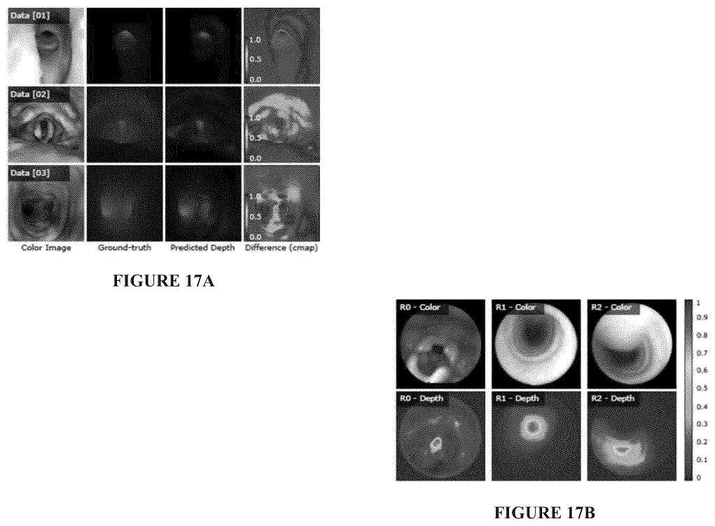

Depth classification system 101 may train evaluation models using ML techniques. The ML techniques may include evaluating the accuracy of existing models. For example, models may be evaluated based on the absolute difference between known values and values obtained via image prediction techniques. Predicted depth images may also be evaluated based on Mean Squared Error (MSE) and Structural Similarity Index Measure (SSIM) (e.g., see FIG. 17A).

Turning back to FIG. 13, depth classification system 101 uses GAN and ML techniques to generate predicted depth images which in turn are converted into point-cloud data for generating dynamic surface estimations. For the select result prediction images shown in FIG. 17A, the MSE scores are: (124.14, 745.98, 409.48) with SSIM scores: (0.89, 0.82, 0.87). For the training dataset: avg-MSE: 510.10 and avg-SSIM: 0.821. This illustrates the GAN model is effective at estimating synthesized data; however, real-world DISE video presents a challenge due to the lower resolution, complex features, and interference. Select GAN depth estimates for the real DISE images (R0-R2) are shown in FIG. 17B.

Various factors contribute to the accuracy and quality of predicted depth images that define airway structural geometry. This includes the visual quality of the synthesized GAN training data, image quality of the DISE sequences, obtaining sufficient Cine MRI data, and the integration of these data sources into an objective model. Cine MRI level analytical quality may be achieved through a monocular stream of images from DISE using the technology disclosed herein.

Due to unknown intrinsic characteristics of cameras (e.g., lens, resolution, field-of-view, image quality, etc.), close proximity of the camera to complex airway structures, and/or patient-specific characteristics, large variances may exist in images generated by endoscopic cameras. As a result, a sufficient range of training data may be necessary to account for degradation of image data quality. For example, the range of training data may comprise a diverse set of images to account for variances in image quality.

Behavioral modeling may be constructed from the dynamic model generation system. For example, quantitative measurements may be obtained from a generated 4D model, providing an evaluation of how the observed surfaces within an airway change and impact flow. Quantitative measurements of the generated model may be obtained and used to identify and evaluate effective flow (e.g., through an upper airway). To achieve an increase in accuracy, imaging may be limited to monocular DISE video (e.g., of the upper airway) and/or measurements may be extracted from prior training data. The prior training data may be obtained from secondary imaging methods (e.g., CT, Cine-MRI, etc.). Thus, enabling quantitative measurements that vary with the behavioral deformation of the airway.

Using a trained GAN model to generate surface models of input images enables the measurement of complex deformation behaviors that may be superimposed on surface models generated from live-streamed video feed. Measurements obtained from live-streamed video may be used to provide immediate feedback (e.g., to an examining physician). Feedback may be in the form of a visual and/or audio alarm, pop-up notification, and the like. Notifications may be surfaced in a user interface of an application.

What is claimed is:

1. A method for dynamically assessing airway deformation comprising:
receiving endoscopic image data of an airway;
generating depth data from the endoscopic image data using a depth prediction model; and
generating a dynamic representation of the airway based on the endoscopic image data and the depth data.

2. The method of claim 1, further comprising:
generating the depth prediction model, wherein generating the depth prediction model comprises:
generating a structural model of an airway from endoscopic image data and MRI data;
generating a training dataset comprising synthetic image data and synthetic depth data generated from the structural model; and
training the depth prediction model from the training dataset.

3. The method of claim 2 wherein the training dataset comprises at least one video sequence, wherein the at least one video sequence comprises the synthetic image data and the synthetic depth data.

4. The method of claim 3 wherein the at least one video sequence further comprises a parameter set, wherein the parameter set comprises one or more of camera exposure data, lighting data, airway surface data, and image artifact data.

5. The method of claim 1, wherein the endoscopic image data comprises a sequence of monocular images.

6. The method of claim 1 wherein the dynamic representation of the airway comprises a sequence of point cloud surface representations, wherein the sequence of point cloud surface representations is generated from the endoscopic image data and the depth data.

7. The method of claim 6 further comprising determining an airway contour at an axial location of the airway from the dynamic representation of the airway.

8. The method of claim 7 further comprising quantifying characteristics of the airway contour, the characteristics comprising a cross-sectional area and a deformation.

9. The method of claim 6 further comprising determining an airway deformation at an axial location of the airway.

10. The method of claim 1 further comprising identifying regions of the airway based on the endoscopic image data and the depth data.

11. The method of claim 1 further comprising identifying regions of the airway that contribute to obstructed breathing based on endoscopic image data and the depth data.

12. A computing apparatus comprising:
one or more computer readable storage media;
one or more processors operatively coupled with the one or more computer readable storage media; and
program instructions stored on the one or more computer readable storage media that, when executed by the one or more processors, direct the computing apparatus to at least:
receive endoscopic image data of an airway;
generate depth data from the endoscopic image data using a depth prediction model; and
generate a dynamic representation of the airway based on the endoscopic image data and the depth data.

13. The computing apparatus of claim 12, wherein the program instructions further direct the computing apparatus to:
generate a structural model of an airway from endoscopic image data and MRI data;
generate a training dataset comprising synthetic image data and synthetic depth data generated from the structural model; and
train the depth prediction model from the training dataset.

14. The computing apparatus of claim 13 wherein the training dataset comprises at least one video sequence, wherein the at least one video sequence comprises the synthetic image data and the synthetic depth data.

15. The computing apparatus of claim 12 wherein the dynamic representation of the airway comprises a sequence of point cloud surface representations, wherein the sequence of point cloud surface representations is generated from the endoscopic image data and the depth data.

16. The computing apparatus of claim 15 wherein the program instructions further direct the computing apparatus to determine an airway contour at an axial location of the airway from the dynamic representation of the airway.

17. One or more computer readable storage media having program instructions stored thereon that, when executed by one or more processors, direct a computing apparatus to at least:
receive endoscopic image data of an airway;
generate depth data from the endoscopic image data using a depth prediction model; and
generate a dynamic representation of the airway based on the endoscopic image data and the depth data.

18. The one or more computer readable storage media of claim 17, wherein the program instructions further direct the computing apparatus to:
generate a structural model of an airway from endoscopic image data and MRI data;
generate a training dataset comprising synthetic image data and synthetic depth data generated from the structural model; and
train the depth prediction model from the training dataset.

19. The one or more computer readable storage media of claim 18 wherein the training dataset comprises at least one video sequence, wherein the at least one video sequence comprises the synthetic image data and the synthetic depth data.

20. The one or more computer readable storage media of claim 19 wherein the dynamic representation of the airway comprises a sequence of point cloud surface representations, wherein the sequence of point cloud surface representations is generated from the endoscopic image data and the depth data.
